(19) United States
(12) Patent Application Publication
(10) Pub. No.: US 2010/0168539 A1
(43) Pub. Date: Jul. 1, 2010

(54) METHOD AND/OR SYSTEM FOR ESTIMATING GLYCATION OF HEMOGLOBIN

(76) Inventors: Cesar C. Palerm, Pasadena, CA (US); Lawrence Shepp, Piscataway, NJ (US); Cun-Hui Zhang, East Brunswich, NJ (US)

Correspondence Address: BERKELEY LAW & TECHNOLOGY GROUP, LLP, 17933 NW Evergreen Parkway, Suite 250, BEAVERTON, OR, 97006, US

(21) Appl. No.: 12/347,778

(22) Filed: Dec. 31, 2008

(51) Int. Cl. A61B 5/145

(52) U.S. Cl. 600/365

(57) ABSTRACT

Disclosed are systems, methods and techniques to estimate an extent of glycation of hemoglobin in a patient. In one particular implementation, although claimed subject matter is not limited in this respect, an estimate of glycation of hemoglobin in a patient may be measured based, at least in part, on blood-glucose measurements obtained from the patient.

### BACKGROUND

1. Field

Subject matter disclosed herein relates to systems, methods and techniques to estimate an extent of glycation of hemoglobin in a patient.

2. Information

The process of glycation is a nonenzymatic addition of glucose to reactive sites in proteins. For example, glycated hemoglobin or glycohemoglobin is a typically characterized adduct and an analyte widely used to monitor glycemic control in diabetic patients. Reactive sites in hemoglobin may include N-terminal valine-amino groups of α-chains, β-chains and ε-amino groups of lysine residues. Hemoglobin A1c (or HbA1c) is one form of glycohemoglobin. Here, such a hemoglobin is irreversibly glycated at one or both N-terminal valine residues of a β-chain of hemoglobin A0. Glycation of hemoglobin in a patient is typically quantified as a percentage of total hemoglobin.

A strong relationship exists between hemoglobin A1c levels in a diabetes patient and risks of micro-vascular complications. Accordingly, hemoglobin A1c measurements have become an integral component of the treatment of diabetes patients. Hemoglobin A1c measurements are typically obtained from a patient through drawing of blood and employing laboratory analysis techniques including centrifuge methods.

### SUMMARY

Briefly, one embodiment relates to a method, system and/or apparatus for estimating a probability distribution associated with the probability of a hemoglobin molecule being glycated at a particular age of said hemoglobin molecule in a patient; and estimating hemoglobin A1c of said patient based, at least in part, on said probability distribution and blood-glucose measurements taken from said patient.

In one particular embodiment, estimating said probability distribution further comprises estimating a rate at which hemoglobin is glycated in said patient. In one particular implementation, estimating said rate at which hemoglobin is glycated in a patient comprises estimating said rate based, at least in part, on hemoglobin A1c measurements taken from blood drawn from said patient. In another particular implementation, estimating said rate further comprises periodically updating said rate based, at least in part, on a least square error estimate from a plurality of hemoglobin A1c measurements. In an alternative implementation, estimating said rate further comprises associating one or more attributes of said patient with a look up table.

In another particular embodiment, estimating said probability distribution comprises estimating said probability based, at least in part, on an exponential probability distribution.

In another particular embodiment, said blood-glucose measurements are obtained at periodic sample intervals.

In another particular embodiment, said blood-glucose measurements are obtained from a blood-glucose sensor implanted in said patient. One particular implementation further includes displaying said estimate of said hemoglobin A1c on a display coupled to said blood-glucose sensor. Another particular implementation includes storing said blood-glucose measurements obtained from said blood-glucose sensor in a memory; and executing a computing platform to estimate said hemoglobin A1c based, at least in part, on said stored blood-glucose measurements.

Particular embodiments may be directed to an article comprising a storage medium including machine-readable instructions stored thereon which, if executed by a computing platform, are directed to enable the computing platform to execute at least a portion of the aforementioned method according to one or more of the particular aforementioned implementations. In other particular embodiments, a sensor adapted generate one or more signals responsive to a blood glucose concentration in a body while a computing platform is adapted to perform the aforementioned method according to one or more of the particular aforementioned implementations based upon the one or more signals generated by the sensor. In one particular implementation, such a computing platform may be associated with a display to display a determined estimate of said hemoglobin A1c

### BRIEF DESCRIPTION OF THE FIGURES

Non-limiting and non-exhaustive features will be described with reference to the following figures, wherein like reference numerals refer to like parts throughout the various figures, in which:

FIG. 1 is a flow diagram illustrating a process for estimating a level of hemoglobin A1c in a patient according to an embodiment;

FIG. 2 is a is a perspective view illustrating a subcutaneous sensor insertion set and telemetered characteristic monitor transmitter device according to an embodiment;

FIG. 3 is an enlarged longitudinal vertical section taken on the line 2-2 of FIG. 2;

FIG. 4 is an enlarged longitudinal sectional of a slotted insertion needle used in an insertion set of FIGS. 2 and 3 according to an embodiment;

FIG. 5 is an enlarged transverse section taken generally on the line 4-4 of FIG. 4;

FIG. 6 is an enlarged transverse section taken generally on the line 5-5 of FIG. 4;

FIG. 7 is an enlarged fragmented sectional view corresponding generally with the encircled region 6 of FIG. 3;

FIG. 8 is an enlarged transverse section taken generally on the line 7-7 of FIG. 3.

FIG. 9A is a top plan and partial cut-away view of a telemetered characteristic monitor transmitter device in accordance with an embodiment;

FIG. 9B is a schematic block diagram of portions of a telemetered characteristic monitor transmitter device in accordance with an embodiment;

FIG. 10 is a schematic block diagram of a characteristic monitor used in accordance with an embodiment; and

FIG. 11 is a schematic block diagram of a telemetered characteristic monitor transmitter and characteristic monitor system in accordance with an embodiment.

### DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, methods, apparatuses or systems that would be known by one of ordinary skill have not been described in detail so as not to obscure claimed subject matter.

Reference throughout this specification to “one embodiment” or “an embodiment” may mean that a particular feature, structure, or characteristic described in connection with a particular embodiment may be included in at least one embodiment of claimed subject matter. Thus, appearances of the phrase “in one embodiment” or “an embodiment” in various places throughout this specification are not necessarily intended to refer to the same embodiment or to any one particular embodiment described. Furthermore, it is to be understood that particular features, structures, or characteristics described may be combined in various ways in one or more embodiments. In general, of course, these and other issues may vary with the particular context of usage. Therefore, the particular context of the description or the usage of these terms may provide helpful guidance regarding inferences to be drawn for that context.

Likewise, the terms, “and,” “and/or,” and “or” as used herein may include a variety of meanings that also is expected to depend at least in part upon the context in which such terms are used. Typically, “or” as well as “and/or” if used to associate a list, such as A, B or C, is intended,to mean A, B, and C, here used in the inclusive sense, as well as A, B or C, here used in the exclusive sense. In addition, the term “one or more” as used herein may be used to describe any feature, structure, or characteristic in the singular or may be used to describe some combination of features, structures or characteristics. Though, it should be noted that this is merely an illustrative example and claimed subject matter is not limited to this example.

As pointed out above, monitoring of HbA1c levels in diabetes patients allows for the control of conditions leading to micro-vascular complications. Such HbA1c levels may be monitored using laboratory analysis techniques applied to blood samples drawn from patients (e.g., centrifuge analysis). Unfortunately, such techniques are costly and typically inconvenience patients by requiring such patients to travel to a laboratory facility to deposit blood for analysis. Also, determining hemoglobin A1c levels in a patient using laboratory analysis including centrifuge techniques incurs cost.

According to an embodiment, although claimed subject matter is not limited in this respect, HbA1c levels in a patient may be estimated based, at least in part, on blood-glucose measurements obtained from the patient. Here, a probability distribution of hemoglobin glycation for a patient may be estimated based, at least in part, on information and/or attributes associated with the patient. As described below, such an HbA1c level may be estimated by application of such blood-glucose measurements to the estimated probability distribution. It should be understood, however, that this is merely an example embodiment, and that claimed subject matter is not limited in this respect.

As discussed below according to a particular implementation, a probability distribution of hemoglobin glycation may be estimated based, at least in part, on blood-glucose samples obtained from the patient over a period of time. However, this is merely one example of how such a probability distribution may be estimated and claimed subject matter is not limited in this respect.

Using blood-glucose measurements to estimate HbA1c levels enables obtaining the use of convenient devices such as blood-glucose sensors for estimating HbA1c levels without the inconvenience and expense of drawing blood for laboratory analysis. In one implementation, measurements from a blood-glucose sensor implanted in a patient may be uploaded to an offline computing platform. Here, such an offline computing platform may execute software to compute an estimate of the patient's HbA1c level based, at least in part, on the uploaded blood-glucose measurements. In another implementation, a microcomputer and/or microcontroller may be integrated with such an implanted blood-glucose sensor to compute such an estimate of the patient's HbA1c level for local display. It should be understood, however, that these are merely example implementations and that claimed subject matter is not limited to these particular implementations.

A1_{c}(t) may represent a percentage of hemoglobin in a patient which is glycated at time t. According to an embodiment, a patient's HbA1c level may be estimated for a time t based, at least in part, on a series of blood-glucose measurements B(s) taken at set intervals backward in time prior to t, where B(t) represents a blood-glucose level in a patient in mg/dl at time t. According to an embodiment, the probability that a particular hemoglobin molecule in a red blood cell alive at time t may be expressed in relation (1) as follows:

$$\begin{matrix}\frac{A 1 c ( 0 )}{100} = P ( glycated ) \\ = {\int }_{0}^{∞} [ P ( glycated \vert age = t ) P ( cell alive at t ) ] / \\ ( mean lifetime of cell ) d t\end{matrix} \qquad ( 1 )$$

According to an embodiment, for simplicity, a cumulative distribution function of a probability that a red blood cell will die by time t may be assumed to be exponential. Here, such a probability distribution function may be represented as follows:

$$F ( t ) = 1 - {e}^{- \frac{t}{μ}} \qquad ( 2 )$$

Where μ is the mean lifetime of a red blood cell. Such a mean lifetime or the distribution of cell life for red blood cells in a given patient may be assumed to be about 120 days, for example. However, a more precise estimate of a mean lifetime may be determined for the particular patient based, for example, on laboratory tests and/or personal attributes of the particular patient.

In a particular embodiment, a probability density function of the residual lifetime of a red blood cell at time t may be expressed as follows:

$$P ( cell alive at t ) = 1 - F ( t ) = 1 - ( 1 - {e}^{- \frac{t}{μ}} ) \\ P ( cell alive at t ) = {e}^{- \frac{t}{μ}} \qquad ( 3 )$$

Derivation of techniques for modeling a residual lifetime may be described by Walter L. Smith in Renewal Theory and Its Ramifications, Journal of the Royal Statistical Society, Series B, Vol. 20, No. 2 (1958), pp. 243-302. Here, relation (3) is merely an example of how a residual lifetime of a red blood cell may be modeled according to a particular implementation. It should be understood, however, that this is merely example of how a residual lifetime of a red blood cell and that claimed subject matter is not limited to any particular technique for modeling a residual lifetime of a red blood cell.

If a red blood cell is still alive in a patient at time t, and a hemoglobin molecule in the red blood cell has not been glycated at time t, the probability that the molecule is glycated in a following interval dt may be assumed to be substantially proportional to B(t)dt. Thus, the probability that the hemoglobin molecule of age t is not glycated at time t=0 may be approximated as e⁻^{₀}^{¹}^{αB(t-s)ds}. Accordingly, the distribution function of the probability that a hemoglobin molecule is glycated at age t may be estimated as:

$$P ( glycated \vert age = t ) = 1 - {e}^{- {\int }_{0}^{t} α B ( t - s ) d s} , \qquad ( 4 )$$

where α represents an estimate of a rate at which hemoglobin in a patient is glycated as expressed in units of dl/(mg min).

Applying distribution functions of relations (3) and (4) to relation (1), A1_{c} may be estimated based, at least in part, on blood-glucose samples B(s) as follows:

$$A {1}_{c} = 100 \times {\int }_{0}^{∞} \frac{1}{μ} {e}^{- \frac{t}{μ}} [ 1 - {e}^{- {\int }_{0}^{t} α B ( t - s ) d s} ] d t . \qquad ( 5 )$$

It should be observed that an estimate of A1_{c} as computed according to relation (5) in the above described embodiment, is also based on α, an estimate of a rate at which hemoglobin in a patient is glycated. It should be understood that such a rate of glycation of hemoglobin in a patient may depend in large part on the particular physiology of the patient. Accordingly, estimate α may be different for different patients as a result of such different physiologies and, in particular embodiments, estimate α may be tailored and/or determined for a patient based upon the patient's unique physiology. Factors affecting α may include, for example, age, gender, heredity and/or genetic effects. Regarding genetic effects, for example, a glucose absorption gradient across a cell membrane of red blood cells of an individual may have significant effects on α for the individual.

In one particular embodiment, a value for estimate α may be determined for a patient based, at least in part, on A1_{c} measurements taken from the patient by, for example, laboratory analysis of drawn blood as discussed above. Here, such an A1_{c} measurement and history of blood-glucose measurements B(s) may be applied to relation (4), to be solved for α. In one example, for the purpose of illustration, a patient may have a constant blood-glucose level of 100 mg/dl and a measured A1_{c} of 0.05 (5%). In this particular example, according to relation (4) the estimate α may determined from solving the following algebraic expression:

$$0.05 = {\int }_{0}^{∞} \frac{1}{μ} {e}^{- \frac{t}{μ}} ( 1 - {e}^{- 100 α t} ) d t .$$

It should be understood, however, that this is merely simple case assuming a constant blood-glucose level of B(s)=B₀=100 mg/dl. In other examples with a time-varying blood-glucose level, historical data for B(s) may be used to evaluate the following algebraic expression to solve for α:

$$.05 = {\int }_{0}^{∞} \frac{1}{μ} {e}^{- \frac{t}{μ}} [ 1 - {e}^{- {\int }_{0}^{t} α B ( t - s ) d s} ] d t$$

In one embodiment, although claimed subject matter is not limited in this respect, a value of α for a patient may be updated based, at least in part, on a history of values for α taken over time. For example, A1c measurements may be taken from a patient by, for example, drawing of blood and performing a laboratory analysis as illustrated above. Here, it can be seen that a value for α may be computed for each such A1c measurement along with a history of blood-glucose measurements obtained from the patient. In a particular embodiment, such a series of values may be computed using, for example, linear regression, weighted averaging and/or the like to determine a more accurate estimate of α for the patient. In other embodiments, estimate a may be selected from look-up tables indexed to characteristics associated with the patient such as age, gender, physical condition (e.g., pregnancy), just to name a few examples.

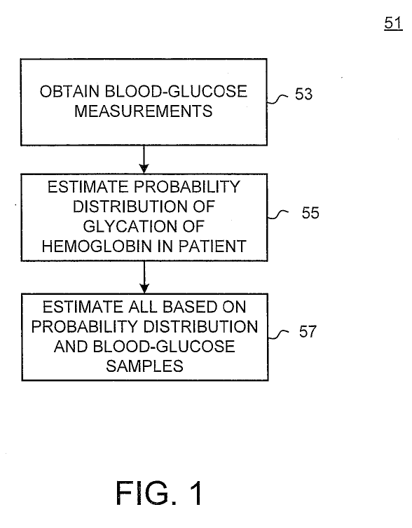

FIG. 1 is a flow diagram of a process 51 to estimate an HbA1c in a patient based, at least in part, on a history of blood-glucose measurements taken from the patient. As discussed above, particular embodiments may be directed to estimating an HbA1c level in a patient, but without the inconvenience of drawing blood for laboratory analysis. At block 53 blood-glucose measurements may be taken from a patient over time. For example, some blood-glucose measurements may be obtained from one or more subcutaneous implanted blood-glucose sensors. Here, for example, such a blood-glucose sensor may obtain blood-glucose measurements on set intervals or periods such as, for example, once every five minutes. It should be understood, however, that this is merely an example of a sample interval for obtaining blood-glucose measurements, and that longer or shorter sample intervals may be used without deviating from claimed subject matter. In one particular implementation, such measurements may be collected, time-stamped and stored in a memory device for use in analysis at a later time as discussed below. In fact, other embodiments may be applied to estimating HbA1c using a history of blood-glucose measurements that are not on set intervals.

Block 55 is directed to estimating a probability distribution function associated with the probability of glycation of hemoglobin in a patient. Such a probability distribution function may be estimated according to relation (4) as described above. Here, the estimate α may be determined using any one of several techniques discussed above such as, for example, according to relation (5) using past A1c measurements obtained from drawn blood using laboratory analysis techniques, or a look-up table.

Relations (4) and (5) above presume a blood-glucose measurement value B(s) that is continuous over 0<s<t. In particular implementations, however, blood-glucose measurements may be obtained at discrete time instances, such as at set time intervals. In a particular implementation where blood-glucose measurements are obtained at set intervals h, relation (4) may be modified as relation (6) as follows:

P(glycated|age=jh)=1−e^{-Σ}^{ₖ₌₀}^{^{j}}^{αhX((j-k)h)}, (6)

where h is a blood-glucose sample step size in minutes, X(m) is one if the m^{th} blood-glucose measurement is available and zero if the m^{th} blood-glucose measurement is not available (e.g., no measurement is taken at m or the m^{th} measurement is determined to be unreliable).

According to an embodiment, block 57 may estimate an A1c level in a patient based, at least in part, on blood-glucose measurements obtained at block 53 and a probability distribution estimated at block 55. Such an estimate may be obtained according to relation (5) as described above. In the particular example implementation described above in connection with obtaining blood-glucose measurements at set intervals, relation (5) may be modified as relation (7) as follows:

$$A 1 c = 100 \times {\sum }_{j = 0}^{L} \frac{{e}^{- \frac{jh}{μ}}}{μ} h ( 1 - {e}^{- {\sum }_{k = 0}^{j} α hX ( ( j - k ) h )} ) , \qquad ( 7 )$$

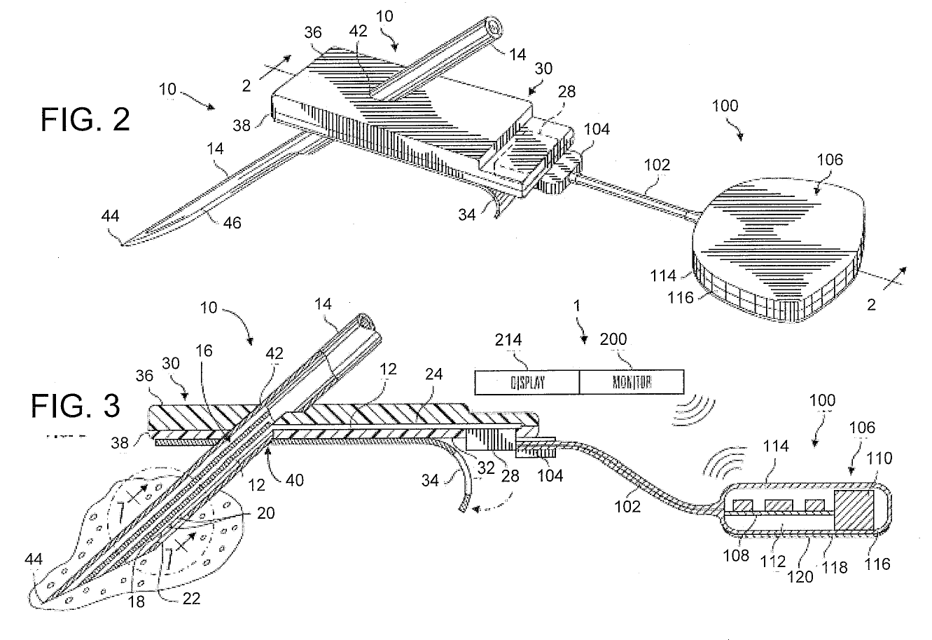

As pointed out above in connection with particular embodiments, HbA1c levels in a patient may be estimated based, at least in part, on blood-glucose measurements taken from a blood glucose sensor which is implanted in the patient. In a particular implementation as shown in FIG. 2, a telemetered characteristic monitor system 1 includes a percutaneous blood-glucose sensor set 10, a telemetered characteristic monitor transmitter device 100 and a characteristic monitor 200. Sensor set 10 may utilize an electrode-type sensor, as described in more detail below. However, in alternative embodiments, other types of blood-glucose sensors, such as chemical based, optical based or the like capable of measuring blood-glucose in a patient may be used without deviating from claimed subject matter. In further alternative embodiments, such blood-glucose sensors may be of a type that is used on the external surface of the skin or placed below the skin layer of the user. In one particular implementation, a surface mounted blood-glucose sensor may utilize interstitial fluid harvested from underneath a patient's skin. Device 100 may include a capability to transmit data in a wireless transmission link. In alternative embodiments, device 100 may include a receiver, or the like, to facilitate two-way communication between sensor set 10 and characteristic monitor 200. Characteristic monitor 200 may utilize transmitted data to determine a characteristic reading. However, in alternative embodiments, characteristic monitor 200 may be replaced with a data receiver, storage and/or transmitting device for later processing of the transmitted data or programming of device 100.

In addition, a relay or repeater 4 may be used in conjunction with device 100 and characteristic monitor 200 to allow a greater separation between device 100 and characteristic monitor 200, as shown in FIG. 11. Also, relay 4 may be capable of providing information obtained by device 100 data from the sensor set 10, as well as other data, to a remote receiver for processing. Such data may also be downloaded through a Communication-Station 8 to a remotely located computer 6 such as a PC, lap top computer, or other like computing platform, over wired or wireless communication links, as shown in FIG. 11. Also, some embodiments may omit Communication Station 8 and use a direct modem and/or wireless connection to computer 6 instead. In further embodiments, device 100 may transmit to an RF programmer, which acts as a relay, or shuttle, for data transmission between sensor set 10 and a PC, lap top computer, Communication-station, a data processor, and/or the like.

Alternative embodiments may include a capability for simultaneous monitoring of multiple sensors and/or include a sensor for multiple measurements. Still further embodiments of device 100 may have and use an input port for direct (e.g., wired) connection to a programming or data readout device and/or be used for calibration of sensor set 10. Here, such a port may be water proof (or water resistant) and/or include a water proof, or water resistant, removable cover.

According to an embodiment, blood-glucose measurements taken from sensor 10 may be wirelessly transmitted to characteristic monitor 200, which may display and log the received blood-glucose measurements. Logged data can be downloaded from characteristic monitor 200 to a computing platform such as a personal computer, laptop, and/or the like, for detailed data analysis. Such analysis may include, for example, estimating a level of HbA1c associated with a patient using techniques discussed above. In further embodiments, one or more buttons (on device 100 or characteristic monitor 200) may be manually selected to record data and events for later analysis, correlation, or the like. In addition, device 100 may include a transmit on/off button for compliance with safety standards and regulations to temporarily suspend transmissions. Further buttons can include a sensor on/off button to conserve power and/or to assist in initializing sensor set 10. Device 100 and characteristic monitor 200 may also be combined with other medical devices to combine other patient data through a common data network and/or telemetry system.

Further embodiments of sensor set 10 may monitor the temperature of sensor set 10, which can then be used to improve calibration of the sensor. For instance, for a glucose sensor, an enzyme reaction activity may have a known temperature coefficient. A relationship between temperature and enzyme activity can be used to adjust the sensor values to more accurately reflect the actual blood-glucose levels. In addition to temperature measurements, an oxygen saturation level can be determined by measuring signals from various electrodes of sensor set 10. Once obtained, an oxygen saturation level may be used in calibration of sensor set 10 due to changes in the oxygen saturation levels, and its effects on the chemical reactions in sensor set 10. For instance, as the oxygen level goes lower the sensor sensitivity may be lowered. An oxygen level can be utilized in calibration of sensor set 10 by adjusting for a change in oxygen saturation. In alternative embodiments, temperature measurements may be used in conjunction with other readings to calibrate a blood-glucose sensor.

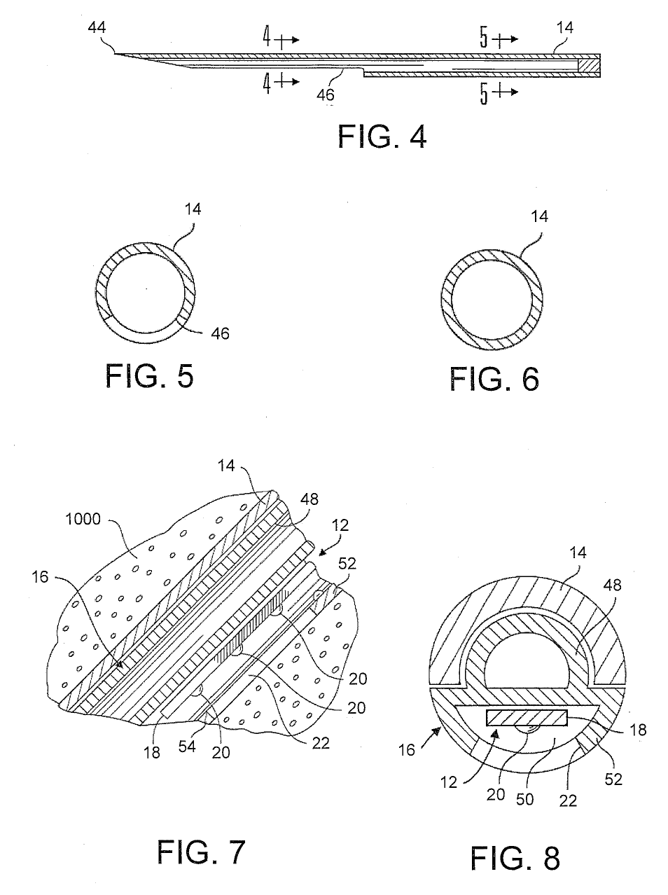

As shown in FIGS. 2 through 8, sensor set 10 is provided for subcutaneous placement of an active portion of a flexible sensor 12 (see FIG. 3), or the like, at a selected site in the body of a patient. A subcutaneous or percutaneous portion of sensor set 10 includes a hollow, slotted insertion needle 14, and a cannula 16. Insertion needle 14 is used to facilitate quick and easy subcutaneous placement of the cannula 16 at the subcutaneous insertion site. Inside the cannula 16 is a sensing portion 18 of the sensor 12 to expose one or more sensor electrodes 20 to the patient's bodily fluids through a window 22 formed in the cannula 16. After insertion, insertion needle 14 is withdrawn to leave the cannula 16 with sensing portion 18 and sensor electrodes 20 in place at the selected insertion site.

In particular embodiments, sensor set 10 may facilitate accurate placement of a flexible thin film electrochemical sensor 12 of the type used for monitoring specific blood parameters representative of a patient's condition. For example, sensor 12 may monitor glucose levels in the patient's body, and may be used in conjunction with automated or semi-automated medication infusion pumps of the external or implantable type as described, for example, in U.S. Pat. Nos. 4,562,751; 4,678,408; 4,685,903 or 4,573,994, to control delivery of insulin to a diabetic patient.

Particular embodiments of flexible electrochemical sensor 12 are constructed in accordance with thin film mask techniques to include elongated thin film conductors embedded or encased between layers of a selected insulative material such as polyimide film or sheet, and membranes. Sensor electrodes 20 at a tip end of the sensing portion 18 are exposed through one of the insulative layers for direct contact with patient blood or other body fluids, if sensing portion 18 (or active portion) of sensor 12 is subcutaneously placed at an insertion site. Sensing portion 18 may be joined to a connection portion 24 (see FIG. 3) that terminates in conductive contact pads, or the like, which are also exposed through one of the insulative layers. In alternative embodiments, other types of implantable sensors, such as chemical based, optical based, or the like, may be used.

As is known in the art, and illustrated schematically in FIG. 3, connection portion 24 and the contact pads may be adapted for a direct wired electrical connection to a suitable monitor 200 for monitoring a user's condition in response to signals derived from sensor electrodes 20. Further description of flexible thin film sensors of this general type are be found in U.S. Pat. No. 5,391,250, entitled METHOD OF FABRICATING THIN FILM SENSORS. According to an embodiment, connection portion 24 may be conveniently connected electrically to the monitor 200 or a telemetered characteristic monitor transmitter 100 by a connector block 28 (or the like) as shown and described in U.S. Pat. No. 5,482,473, entitled FLEX CIRCUIT CONNECTOR. Thus, in accordance with particular embodiments, subcutaneous sensor sets 10 may be configured or formed to work with either a wired or a wireless characteristic monitor system.

A proximal portion of sensor 12 is mounted in a mounting base 30 adapted for placement onto the skin of a user. As shown, mounting base 30 comprises a pad having an underside surface coated with a suitable pressure sensitive adhesive layer 32, with a peel-off paper strip 34 normally provided to cover and protect adhesive layer 32, until sensor set 10 is ready for use. As shown in FIGS. 2 and 3, mounting base 30 includes upper and lower layers 36 and 38, with connection portion 24 of flexible sensor 12 being sandwiched between layers 36 and 38. Connection portion 24 has a forward section joined to active sensing portion 18 of sensor 12, which is folded angularly to extend downwardly through a bore 40 formed in lower base layer 38. In particular embodiments, adhesive layer 32 includes an anti-bacterial agent to reduce the chance of infection; however, alternative embodiments may omit the agent. In the illustrated embodiment, the mounting base is generally rectangular, but alternative embodiments may be other shapes, such as circular, oval, hour-glass, butterfly, irregular, or the like.

Insertion needle 14 is adapted for slide-fit reception through a needle port 42 formed in the upper base layer 36 and further through lower bore 40 in lower base layer 38. As shown, insertion needle 14 has a sharpened tip 44 and an open slot 46 which extends longitudinally from tip 44 at the underside of needle 14 to a position at least within bore 40 in the lower base layer 36. Above mounting base 30, insertion needle 14 may have a full round cross-sectional shape, and may be closed off at a rear end of needle 14. Further description of the needle 14 and the sensor set 10 are found in U.S. Pat. Nos. 5,586,553 and 5,954,643.

Cannula 16 is further illustrated in FIGS. 7 and 8, and includes a first portion 48 having partly-circular cross-section to fit within the insertion needle 14 that extends downwardly from mounting base 30. In alternative embodiments, first portion 48 may be formed with a solid core; rather than a hollow core. In particular embodiments, cannula 16 is constructed from a suitable medical grade plastic or elastomer, such as polytetrafluoroethylene, silicone, and/or the like. Cannula 16 also defines an open lumen 50 in a second portion 52 for receiving, protecting and guideably supporting sensing portion 18 of sensor 12. Cannula 16 has one end fitted into bore 40 formed in lower layer 38 of mounting base 30, and cannula 16 is secured to mounting base 30 by a suitable adhesive, ultrasonic welding, snap fit or other selected attachment method. From mounting base 30, cannula 16 extends angularly downwardly with first portion 48 nested within insertion needle 14, and terminates before needle tip 44. At least one window 22 is formed in lumen 50 near implanted end 54, in general alignment with sensor electrodes 20, to permit direct electrode exposure to the user's bodily fluid when sensor 12 is subcutaneously placed. Alternatively, a membrane can cover this area with a porosity that controls rapid diffusion of glucose through the membrane.

As shown in FIGS. 2, 3 and 9A, telemetered characteristic monitor transmitter 100 is coupled to sensor set 10 by a cable 102 through a connector 104 that is electrically coupled to connector block 28 of connector portion 24 of sensor set 10. In alternative embodiments, cable 102 may be omitted, and telemetered characteristic monitor transmitter 100 may include an appropriate connector (not shown) for direct connection to connector portion 24 of sensor set 10, or sensor set 10 may be modified to have connector portion 24 positioned at a different location, such as for example, on the top of sensor set 10 to facilitate placement of the telemetered characteristic monitor transmitter over subcutaneous sensor set 10. This may reduce an amount of skin surface covered or contacted by medical devices, and tend to reduce movement of sensor set 10 relative to telemetered characteristic monitor transmitter 100. In further alternative embodiments, cable 102 and connector 104 may be formed as add-on adapters to fit different types of connectors on different types or kinds of sensor sets. The use of adapters may facilitate adaptation of telemetered characteristic monitor transmitter 100 to work with a wide variety of sensor systems. In further embodiments, telemetered characteristic monitor transmitter 100 may omit cable 102 and connector 104 and is instead optically couple with an implanted sensor, in the subcutaneous, dermal, sub-dermal, inter-peritoneal or peritoneal tissue, to interrogate the implanted sensor using visible, and/or IR frequencies, either transmitting to and receiving a signal from the implanted sensor or receiving a signal from the implanted sensor.

Telemetered characteristic monitor 100 (also known as Potentiostat Transmitter Device) includes a housing 106 that supports a printed circuit board 108, batteries 110, antenna 112, and cable 102 with connector 104. In particular embodiments, housing 106 is formed from an upper case 114 and a lower case 116 that are sealed with an ultrasonic weld to form a waterproof (or resistant) seal to permit cleaning by immersion (or swabbing) with water, cleaners, alcohol or the like. In particular embodiments, upper and lower case 114 and 116 are formed from a medical grade plastic. However, in alternative embodiments, upper case 114 and lower case 116 may be connected together by other methods, such as snap fits, sealing rings, RTV (silicone sealant) and bonded together, or the like, or formed from other materials, such as metal, composites, ceramics, or the like. In other embodiments, the separate case can be eliminated and the assembly is simply potted in epoxy or other moldable materials that is compatible with the electronics and reasonably moisture resistant. In particular embodiments, housing 106 may be disk or oval shaped. However, in alternative embodiments, other shapes, such as hour glass, rectangular or the like, may be used. Particular implementations of housing 106 may be sized in the range of 2.0 square inches by 0.35 inches thick to reduce weight, discomfort and the noticeability of telemetered characteristic monitor transmitter 100 on the body of the patient. However, larger or smaller sizes, such as 1.0 square inches and 0.25 inches thick or less, and 3.0 square inches and 0.5 inches thick or more, may be used. Also, the housing may simply be formed from potted epoxy, or other material, especially if the battery life relative to the device cost is long enough, or if the device is rechargeable.

As shown, lower case 116 may have an underside surface coated with a suitable pressure sensitive adhesive layer 118, with a peel-off paper strip 120 normally provided to cover and protect adhesive layer 118, until the sensor set telemetered characteristic monitor transmitter 100 is ready for use. In preferred implementations, adhesive layer 118 includes an anti-bacterial agent to reduce the chance of infection; however, alternative embodiments may omit the agent. In further alternative embodiments, adhesive layer 118 may be omitted and telemetered characteristic monitor transmitter 100 is secured to the body by other methods, such as an adhesive overdressing, straps, belts, clips or the like.

In particular implementations, cable 102 and connector 104 may be similar to (but not necessarily identical to) shortened versions of a cable and connector that are used to provide a standard wired connection between the sensor set 10 and characteristic monitor 200. This may allow the telemetered characteristic monitor transmitter 100 to be used with existing sensor sets 10, and avoid the necessity to re-certify connector portion 24 of sensor set 10 for use with a wireless connection. Cable 102 may also include a flexible strain relief portion (not shown) to reduce strain on the sensor set 10 and prevent movement of the inserted sensor 12, which can lead to discomfort or dislodging of the sensor set 10. The flexible strain relief portion is intended to minimize sensor artifacts generated by user movements that might cause the sensing area of sensor set 10 to move relative to the body tissues in contact with the sensing area of sensor set 10.

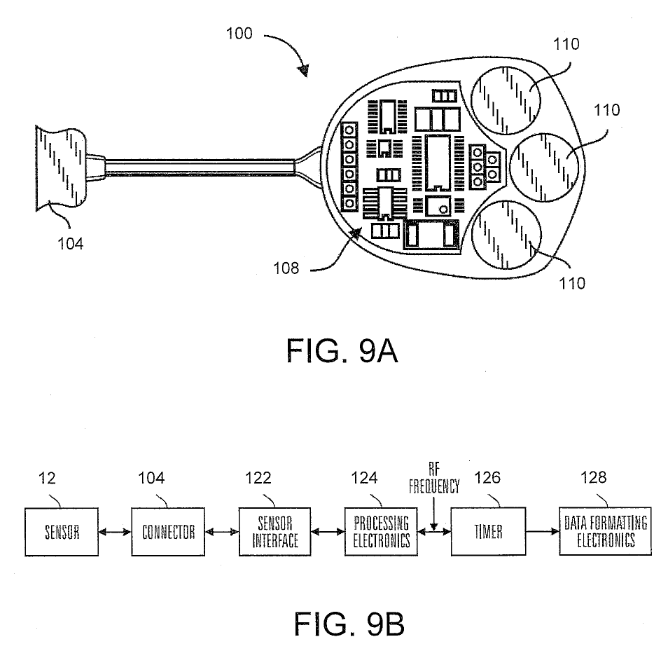

Printed circuit board 108 of telemetered characteristic monitor transmitter 100 may include a sensor interface 122, processing electronics 124, timers 126, and data formatting electronics 128, as shown in FIG. 9B. In particular implementations, the sensor interface 122, processing electronics 124, timers 126, and data formatting electronics 128 are formed as separate semiconductor chips; however, alternative embodiments may combine the various semiconductor chips into a single customized semiconductor chip. Sensor interface 122 connects with cable 102 that is connected with sensor set 10. In particular embodiments, sensor interface 122 is permanently connected to the cable 102. However, in alternative embodiments, sensor interface 122 may be configured in the form of a jack to accept different types of cables that provide adaptability of the telemetered characteristic monitor transmitter 100 to work with different types of sensors and/or sensors placed in different locations of the user's body. In particular embodiments, printed circuit board 108, and associated electronics, are capable of operating in a temperature range of 0° C. and 50° C. However, larger or smaller temperature ranges may be used.

In particular implementations, a battery assembly may use a weld tab design to connect power to the system. For example, it can use a series silver oxide 357 battery cells 110, or the like. However, it is understood that different battery chemistries may be used, such as lithium based chemistries, alkaline batteries, nickel metalhydride, or the like, and different numbers of batteries can be used. In further embodiments, sensor interface 122 may include circuitry and/or a mechanism for detecting connection to sensor set 10. This may provide a capability to save power and to more quickly and efficiently start initialization of sensor set 10. In particular embodiments, batteries 110 may have a life in the range of 3 months to 2 years, and provide a low battery warning alarm. Alternative embodiments may provide longer or shorter battery lifetimes, or include a power port, solar cells or an inductive coil to permit recharging of rechargeable batteries in telemetered characteristic monitor transmitter 100.

In particular implementations, telemetered characteristic monitor transmitter 100 may provide power through cable 102 and cable connector 104 to sensor set 10. Such power may be used to monitor and drive the sensor set 10. Such a power connection may also initialization of sensor 12, if sensor 12 is first placed under the skin. Such use of an initialization process may reduce the time for sensor 12 stabilization from several hours to an hour or less. Such an initialization procedure may employ a two step process. First, a high voltage (e.g., between 1.0-1.2 volts—although other voltages may be used) is applied to sensor 12 for one to two minutes (although different time periods may be used) to allow sensor 12 to stabilize. Then, a lower voltage (e.g., between 0.5-0.6 volts—although other voltages may be used) is applied for the remainder of the initialization process (e.g., 58 minutes or less). Other stabilization/initialization procedures using differing currents, currents and voltages, different numbers of steps, or the like, may be used. Other embodiments may omit the initialization/stabilization process, if not required by sensor 12 or if timing is not a factor.

At completion of such a stabilizing process, a reading may be transmitted from sensor set 10 and the telemetered characteristic monitor transmitter 100 to characteristic monitor 200, and then the user may input a calibrating glucose reading into characteristic monitor 200. In alternative embodiments, a fluid containing a known value of glucose may be injected into the site around the sensor set 10, and then the reading is sent to the characteristic monitor 200 and the user inputs the known concentration value, presses a button (not shown) or otherwise instructs the monitor to calibrate using the known value. During such a calibration process, telemetered characteristic monitor transmitter 100 may check to determine whether sensor set 10 is still connected. If the sensor set 10 is no longer connected, telemetered characteristic monitor transmitter 100 may abort the stabilization process and sound an alarm (or send a signal to the characteristic monitor 200 to sound an alarm).

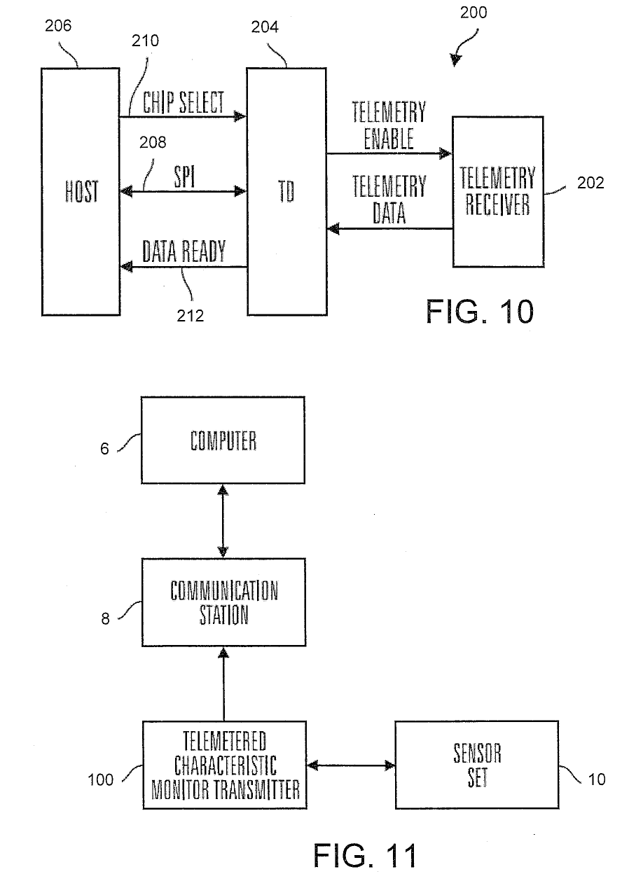

As shown in FIG. 10, characteristic monitor 200 includes a telemetry receiver 202, a Telemetry Decoder (TD) 204 and a host micro-controller (Host) 206 for communication with the telemetered characteristic monitor transmitter 100. TD 204 may decode a received telemetry signal from the transmitter device and forward the decoded signal to Host 206. Host 206 may comprise a microprocessor for data reduction, data storage, user interface, or the like. Telemetry receiver 202 may receive characteristic data (e.g., blood-glucose data) from the telemetered characteristic monitor transmitter, and pass it to the TD 204 for decoding and formatting. After complete receipt of the data by TD 204, such data may be transferred to Host 206 for processing. Such processing at Host 206 may include calibration, based upon user entered characteristic readings (e.g., blood glucose readings). Also, host 206 may be adapted to compute an estimate of hemoglobin A1c levels using one or more techniques described above. Host 206 may also provides for storage of historical characteristic data, and can download the data to a personal computer, lap-top, or the like, via a com-station, wireless connection, modem or the like. For example, in particular embodiments, the counter electrode voltage may be included in the message from telemetered characteristic monitor transmitter 100 and used as a diagnostic signal. A raw current signal may have values ranging from 0 to 999, which represents sensor electrode current in the range between 0.0 to 99.9 nanoAmperes, and is converted to characteristic values, such as glucose values in the range of 40 to 400 mg/dl. However, in alternative embodiments, larger or smaller ranges may be used. The values are then displayed on the characteristic monitor 200 or stored in data memory for later recall.

Characteristic monitor 200 may also include circuitry in TD 204 to uniquely mate it to an identified telemetered characteristic monitor transmitter 100. In particular embodiments, an identification number associated with a particular telemetered characteristic monitor transmitter 100 may be entered manually by a patient using keys located on characteristic monitor 200. In alternative embodiments, a characteristic monitor 200 includes a “learn ID” mode. Here, such a “learn ID” mode may be suited for the home environment, since multiple telemetered characteristic monitor transmitters 100, typically encountered in a hospital setting, are less likely to cause confusion in the characteristic monitor 200 if it attempts to learn an ID code. In addition, characteristic monitor 200 may include an ability to learn or be reprogrammed to work with a different (or replacement) telemetered characteristic monitor transmitter 100.

In particular embodiments, characteristic monitor 200 may utilize a two processor system, in which Host 206 is the master processor and TD 204 is a slave processor dedicated to telemetry processing.

In alternative embodiments, TD 204 and Host 206 may be combined together in a single semiconductor device to obviate the need for dual processors and to reduce the space needed for the electronics. In further embodiments, functions of the TD 204 and Host 206 may be allocated differently between or among one or more processors.

As shown in FIG. 3, characteristic monitor 200 may include a display 214 that is used to display the results of the measurement received from sensor 18 in sensor set 10 via telemetered characteristic monitor transmitter 100. Results and information displayed may include, but not be limited to, trending information of the characteristic (e.g., rate of change of blood-glucose), graphs of historical data, average characteristic levels (e.g., glucose), hemoglobin A1c levels and/or the like. Alternative embodiments may include an ability to scroll through the data. Display 214 may also be used with buttons (not shown) on the characteristic monitor to program or update data in characteristic monitor 200.

In one implementation, characteristic monitor 200 may be powered by batteries (not shown). For example, a plurality of silver oxide batteries may be used. However, it is understood that different battery chemistries may be used, such as lithium based, alkaline based, nickel metalhydride, or the like, and different numbers of batteries can be used.

In further embodiments, characteristic monitor 200 may be replaced by a different device. For example, in one embodiment, telemetered characteristic monitor transmitter 100 communicates with an RF programmer (not shown) that is also used to program and obtain data from an infusion pump or the like. Such an RF programmer may also be used to update and program the transmitter 100, if the transmitter 100 includes a receiver for remote programming, calibration or data receipt. Such an RF programmer can be used to store data obtained from sensor 18 and then provide it to either an infusion pump, characteristic monitor, computer or the like for analysis. In further embodiments, the transmitter 100 may transmit the data to a medication delivery device, such as an infusion pump or the like, as part of a closed loop system. This may allow the medication delivery device to compare sensor results with medication delivery data and either sound alarms when appropriate or suggest corrections to the medication delivery regimen. In particular embodiments, transmitter 100 may include a transmitter to receive updates or requests for additional sensor data. An example of one type of RF programmer can be found in U.S. Pat. No. 6,554,798.

In use, sensor set 10 may permit quick and easy subcutaneous placement of sensing portion 18 at a selected site within the body of the user. More specifically, the peel-off strip 34 (see FIG. 3) is removed from the mounting base 30, at which time the mounting base 30 can be pressed onto and seated upon the patient's skin. During this step, insertion needle 14 pierces the patient's skin and carries the protective cannula 16 with sensing portion 18 to the appropriate subcutaneous placement site. During insertion, cannula 16 provides a stable support and guide structure to carry flexible sensor 12 to a desired placement site. While sensor 12 is subcutaneously placed, with the mounting base 30 seated upon the user's skin, insertion needle 14 can be slidably withdrawn from the user. During this withdrawal step, insertion needle 14 slides over the first portion 48 of protective cannula 16, leaving sensing portion 18 with electrodes 20 directly exposed to the user's body fluids via window 22. Further description of needle 14 and sensor set 10 are found in U.S. Pat. Nos. 5,586,553; 5,954,643; and 5,951,521.

Next, connection portion 24 of the sensor set 10 may be connected to cable 102 of telemetered characteristic monitor transmitter 100, so that sensor 12 can then be used over a prolonged period of time for taking blood chemistry measurements or other characteristic readings, such as blood glucose readings in a diabetic patient. Particular embodiments of the telemetered characteristic monitor transmitter 100 detect the connection of sensor 12 to activate telemetered characteristic monitor transmitter 100. For instance, connection of sensor 12 may activate a switch or close a circuit to turn telemetered characteristic monitor transmitter 100 on. Use of a connection detection provides the capability to maximize the battery and shelf life of the telemetered characteristic monitor transmitter prior to use, such as during manufacturing, test and storage. Alternative embodiments may utilize an on/off switch (or button) on telemetered characteristic monitor transmitter 100.

After a sensor set 10 has been used for a period of time, it may be replaced. Here, a sensor set 10 may be disconnected from the cable 102 of telemetered characteristic monitor transmitter 100. In particular embodiments, telemetered characteristic monitor transmitter 100 may be removed and posited adjacent the new site for a new sensor set 10. In alternative embodiments, a patient does not need to remove transmitter 100. A new sensor set 10 and sensor 12 are attached to transmitter 100 and connected to the user's body. Monitoring then continues, as with the previous sensor 12. If telemetered characteristic monitor transmitter 100, is to be replaced, transmitter 100 may be disconnected from sensor set 10 and the patient's body. The user then connects a new transmitter 100, and reprograms the characteristic monitor (or learns) to work with the new transmitter 100. Monitoring then continues, as with the previous sensor 12.

Unless specifically stated otherwise, as apparent from the following discussion, it is appreciated that throughout this specification discussions utilizing terms such as “processing”, “computing”, “calculating”, “determining”, “estimating”, “selecting”, “weighting”, “identifying”, “obtaining”, “representing”, “receiving”, “transmitting”, “storing”, “analyzing”, “creating”, “contracting”, “associating”, “updating”, or the like refer to the actions or processes that may be performed by a computing platform, such as a computer or a similar electronic computing device, that manipulates or transforms data represented as physical, electronic or magnetic quantities or other physical quantities within the computing platform's processors, memories, registers, or other information storage, transmission, reception or display devices. Accordingly, a computing platform refers to a system or a device that includes the ability to process or store data in the form of signals. Thus, a computing platform, in this context, may comprise hardware, software, firmware or any combinations thereof. Further, unless specifically stated otherwise, a process as described herein, with reference to flow diagrams or otherwise, may also be executed or controlled, in whole or in part, by a computing platform.

It should be noted that, although aspects of the above system, method, or process have been described in a particular order, the specific order is merely an example of a process and claimed subject matter is of course not limited to the order described. It should also be noted that the systems, methods, and processes described herein, may be capable of being performed by one or more computing platforms. In addition, the methods or processes described herein may be capable of being stored on a storage medium as one or more machine readable instructions, that if executed may enable and/or client a computing platform to perform one or more actions. “Storage medium” as referred to herein relates to media capable of storing information or instructions which may be operated on, or executed by, by one or more machines. For example, a storage medium may comprise one or more storage devices for storing machine-readable instructions or information. Such storage devices may comprise any one of several media types including, for example, magnetic, optical or semiconductor storage media. For further example, one or more computing platforms may be adapted to perform one or more of the processed or methods in accordance with claimed subject matter, such as the methods or processes described herein. However, these are merely examples relating to a storage medium and a computing platform and claimed subject matter is not limited in these respects.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all aspects falling within the scope of appended claims, and equivalents thereof.

What is claimed is:

1. A method comprising:
estimating a probability distribution associated with a probability of a hemoglobin molecule being glycated at a particular age of said hemoglobin molecule in a patient; and
estimating hemoglobin A1c of said patient based, at least in part, on said probability distribution and blood-glucose measurements taken from said patient.

2. The method of claim 1, wherein said estimating said probability distribution further comprises estimating a rate at which hemoglobin is glycated in said patient.

3. The method of claim 2, wherein said estimating said rate at which hemoglobin is glycated in a patient comprises estimating said rate based, at least in part, on hemoglobin A1c measurements taken from blood drawn from said patient.

4. The method of claim 3, wherein said estimating said rate further comprises periodically updating said rate based, at least in part, on a least square error estimate from a plurality of hemoglobin A1c measurements.

5. The method of claim 2, wherein said estimating said rate further comprises associating one or more attributes of said patient with a look up table.

6. The method of claim 1, wherein said estimating said probability distribution comprises estimating said probability based, at least in part, on an exponential probability distribution.

7. The method of claim 1, wherein said blood-glucose measurements are obtained at periodic sample intervals.

8. The method of claim 1, wherein said blood-glucose measurements are obtained from a blood-glucose sensor implanted in said patient.

9. The method of claim 8, and further comprising displaying said estimate of said hemoglobin A1c on a display coupled to said blood-glucose sensor.

10. The method of claim 8, and further comprising:
storing said blood-glucose measurements obtained from said blood-glucose sensor in a memory; and
executing a computing platform to estimate said hemoglobin A1c based, at least in part, on said stored blood-glucose measurements.

11. An apparatus comprising:
means for estimating a probability distribution associated with a probability of a hemoglobin molecule being glycated at a particular age of said hemoglobin molecule in a patient; and
means for estimating hemoglobin A1c of said patient based, at least in part, on said probability distribution and blood-glucose measurements taken from said patient.

12. The apparatus of claim 11, wherein said means for estimating said probability distribution further comprises means for estimating a rate at which hemoglobin is glycated in said patient.

13. The apparatus of claim 12, wherein said means for estimating said rate at which hemoglobin is glycated in a patient comprises means for estimating said rate based, at least in part, on hemoglobin A1c measurements taken from blood drawn from said patient.

14. The apparatus of claim 13, wherein said means for estimating said rate further comprises means for updating said rate based, at least in part, on a least square error estimate from a plurality of hemoglobin A1c measurements.

15. The apparatus of claim 12, wherein said means for estimating said rate further comprises means for associating one or more attributes of said patient with a look up table.

16. The apparatus of claim 11, wherein said means for estimating said probability distribution comprises means for estimating said probability based, at least in part, on an exponential probability distribution.

17. The apparatus of claim 11, wherein said blood-glucose measurements are obtained at periodic sample intervals.

18. The apparatus of claim 11, wherein said blood-glucose measurements are obtained from a blood-glucose sensor implanted in said patient.

19. The apparatus of claim 18, and further comprising means for displaying said estimate of said hemoglobin A1c on a display coupled to said blood-glucose sensor.

20. An article comprising:
a storage medium, said storage medium comprising machine-readable instructions stored thereon which, if executed by a computing platform, are adapted to direct said computing platform to:
estimate a probability distribution associated with a probability of a hemoglobin molecule being glycated at a particular age of said hemoglobin molecule in a patient; and
estimate hemoglobin A1c of said patient based, at least in part, on said probability distribution and blood-glucose measurements taken from said patient.

21. The article of claim 20, wherein said instructions, if executed by said computing platform, are further adapted to direct said computing platform to estimate said probability distribution based, at least in part, on an estimated rate at which hemoglobin is glycated in said patient.

22. The article of claim 21, wherein said instructions, if executed by said computing platform, are further adapted to direct said computing platform to determine said estimated rate at which hemoglobin is glycated in a patient based, at least in part, on hemoglobin A1c measurements taken from blood drawn from said patient.

23. The article of claim 22, wherein said instructions, if executed by said computing platform, are further adapted to direct said computing platform to update said estimated rate based, at least in part, on a least square error estimate from a plurality of hemoglobin A1c measurements.

24. The article of claim 21, wherein said instructions, if executed by said computing platform, are further adapted to direct said computing platform to estimating said rate by associating one or more attributes of said patient with a look up table.

25. The article of claim 21, wherein said estimating said probability distribution comprises estimating said probability based, at least in part, on an exponential probability distribution.

26. The article of claim 20, wherein said blood-glucose measurements are obtained at periodic sample intervals.

27. The article of claim 20, wherein said blood-glucose measurements are obtained from a blood-glucose sensor implanted in said patient.

28. The article of claim 20, wherein said instructions, if executed by said computing platform, are further adapted to direct said computing platform to initiate display of said estimate of said hemoglobin A1c on a display coupled to said blood-glucose sensor.

29. An apparatus comprising:
a computing platform, said computing platform being adapted to:
estimate a probability distribution associated with a probability of a hemoglobin molecule being glycated at a particular age of said hemoglobin molecule in a patient; and
estimate hemoglobin A1c of said patient based, at least in part, on said probability distribution and blood-glucose measurements taken from said patient.

30. The apparatus of claim 29, and further comprising a blood-glucose sensor adapted to be implanted in said patient to obtain said blood-glucose measurements.

31. The apparatus of claim 30, and further comprising a display coupled to said blood-glucose sensor to display said estimated hemoglobin A1c.
